(19) United States
(12) Patent Application Publication
(10) Pub. No.: US 2009/0275559 A1
(43) Pub. Date: Nov. 5, 2009

(54) Chronotherapeutic formulations of modified-release calcium channel blocker anti-hypertensive drugs in combination with other anti-hypertensive drugs for 24-hour optimal treatment of hypertension, nocturnal hypertension, and/or hypertension with angina

(75) Inventors: Stephen J. Petti, Troy, NY (US); Samn Raffaniello, Troy, NY (US)

Correspondence Address: William M. Brown, 3610 Cherryhill Drive, Orlando, FL, 32822, US

(73) Assignee: Blue Note Pharmaceuticals, Inc., Troy, NY (US)

(21) Appl. No.: 12/432,848

(22) Filed: Apr. 30, 2009

Related U.S. Application Data
(60) Provisional application No. 61/125,914, filed on Apr. 30, 2008.

(51) Int. Cl. A61K 31/554; A61K 31/55; A61K 31/546; A61K 31/4422; A61K 31/4155

(52) U.S. Cl. 514/211.07; 514/212.07; 514/222.5; 514/356; 514/381

(57) ABSTRACT

The present invention relates to formulations of anti-hypertensive drugs. The present invention includes a modified-release formulation of diltiazem hydrochloride that is suitable for once-daily use and which provides delivery of drug either in the early morning hours, or overnight, so as to blunt the natural rise in blood pressure (BP) and heart rate (HR) in the morning and to reduce the slope of the increase in BP in patients with elevated BP (hypertension).

This application claims the benefit of priority of U.S. Provisional Application Ser. No. 61/125,914, filed on Apr. 30, 2008. The entirety of that provisional application is incorporated herein by reference.

### FIELD OF THE INVENTION

The present invention relates to novel chronotherapeutic formulations of anti-hypertensive drugs administered in combination form, as a single therapeutic product. In particular, the invention includes a new modified-release formulation of diltiazem hydrochloride combined in a single product with one or more other anti-hypertensive agents, such as angiotensin-converting enzyme (ACE) inhibitors (ACE-Is), angiotensin receptor blockers (ARBs) and/or diuretics, that is suitable for once-daily dosing (e.g., orally) and which, moreover, provides for the chronotherapeutically patterned delivery of drugs over a 24-hour period, with specific release characteristics that address the early morning pre-waking and immediate post-waking surges in blood pressure (BP), so as to blunt this natural rise, reducing the slope of the increase in BP in patients with elevated BP (hypertension) and reduce overnight hypertension in those hypertensive patients whose BP does not naturally decrease in the overnight hours (approximately one in five hypertensive patients, known as “non-dippers,” a condition also known as “nocturnal hypertension”). The timing of the release of the new modified-release diltiazem component of the combination drug product is also expected to have a positive effect on heart rate (HR) and the rate pressure product (systolic blood pressure×heart rate/100, RPP), in addition to BP, all of which are associated with (or, in the case of RPP, a calculated surrogate measure of oxygen demand, predictive of) the development of target organ damage and serious cardiovascular events and disease outcomes. Patterned, timed-release of combinations of anti-hypertensive drugs is also expected to provide broader and more effective 24-hour plasma concentration coverage, with the effect of better addressing the significant numbers of hypertensive patients who do not respond to single or even multiple administration of anti-hypertensive drugs (resistant hypertension). Other embodiments of the invention include the use of a dihydropyridine calcium channel blocker, such as amlodipine besylate (amlodipine), in lieu of the diltiazem (non-dihydropyridine) CCB component of a combination product.

### BACKGROUND OF THE INVENTION

The existence of circadian rhythms in cardiovascular disease is well established. It is also known that heart rate and blood pressure normally peak during the morning hours and reach a nadir in the late evening, around bedtime. The incidence of myocardial infarction, stroke, sudden cardiac death, and myocardial ischemia increases during the early-morning hours. Angina attacks occur in a diurnal cycle; their occurrence is common in the hours shortly after an individual begins activity, after waking.

Based on these relationships, researchers have begun to apply the science of chronotherapeutics, or the timing of drug effect with biologic need, to improve cardiovascular outcomes. Traditional treatment regimens for conditions associated with circadian variation typically do not account for circadian fluctuations in disease activity.

Chronotherapeutic regimens are intended to provide pharmacological intervention at the most appropriate time point(s), in accordance with circadian rhythms. The concept of chronotherapeutics in treating cardiovascular diseases includes dosing traditional agents at specific times throughout the day, the development of new agents, and the development of chronotherapeutic formulations and combinations of drugs with special release mechanisms targeted at inducing the greatest effect during the pre- and post-waking morning surge in heart rate (HR) and blood pressure (BP) and/or, in select populations, during sleep.

Chronotherapeutic agents, such as Blue Note Pharmaceuticals' proprietary BNP 32,762 modified-release diltiazem formulation (Chronizem™; see U.S. Pat. No. 6,162,463), are specifically intended to provide peak plasma concentrations when their effect is most needed (e.g., in the early morning hours). Further, the lowest concentrations of drugs typically occur at night, when HR and BP are typically lowest and, consequently, cardiovascular events are least likely to occur. However, special consideration also needs to be given to overnight levels of the drug(s) for nocturnal hypertensives, and for all hypertensives in the pre- and post-waking hours, where HR and BP (and the calculated RPP, a measure of myocardial oxygen demand and a surrogate predictor of target organ damage and cardiovascular events) rise. These latter considerations are not adequately addressed by current anti-hypertensive drug formulations.

In normo- and hypertensives, blood pressure (BP) normally “dips” ˜10-20% overnight. However, about one in five patients with systemic hypertension has a 24-h BP profile characterized by a blunted or no nocturnal decline in BP (so-called “non-dippers”). Hypertensive patients whose nocturnal decline in BP is <10% (i.e., non-dippers) have greater levels of target organ damage and cardiovascular events compared with “dipper” patients, who have a normal decline in BP during sleep. Thus, it is especially important to achieve adequate reductions in BP with anti-hypertensive therapy in non-dippers.

There have been theoretical concerns with excessive reduction of nocturnal BP; however, a study by White et al. (1997) (Am. J. Cardiol. 80, 469-474) showed that in patients with relatively-normal-to-high-normal BPs during sleep, an HS (bedtime)-administered calcium channel blocker (CCB) did not excessively lower BP, even at high doses. These findings suggest the likely safety of diltiazem MR agents in patients who do have reductions in nighttime BP before initiation of therapy, as was concluded by White for verapamil, the CCB examined, and that such patients (dippers) would also benefit from the moderating effect on the “normal” slope of the rise in BP in the pre- and post-waking morning hours (i.e., any reduction in slope of the rise would have a potentially beneficial effect on potential target organ damage and potential cardiovascular event outcome), and its effect on myocardial oxygen demand (as assessed by RPP).

Ambulatory blood pressure monitoring (ABPM) measurements have demonstrated a close correlation between BP and target organ damage and CV events, including MI, stroke, and CV mortality. BP typically rises in the morning, beginning in the period several hours immediately pre-waking, and peaking in the period several hours post-waking. There is subsequently a small post-prandial valley, and, eventually (in dippers), a deeper descent during nocturnal rest.

Under certain pathophysiological conditions, the normal nocturnal BP decline (dipping) may be reduced or even reversed. Subjects with a diminished nocturnal BP decline (non-dippers) have a significantly worse prognosis for the development of related target organ damage and cardiovascular events than those with a normal dipper pattern. In particular, the non-dipper circadian BP pattern represents an elevated risk for left ventricular hypertrophy, CV disease, microalbuminuria, CHF, vascular dementia, and MI. The circadian BP profile has direct implications for improving the delivery of anti-hypertensive drug therapies and the qualification of patients for medication trials and assessments (Hermida et al., Advanced Drug Delivery Reviews 2007; 59: 904-922).

A study by Ben-Dov et al., at Hadassah-Hebrew University Medical Center, Jerusalem, examined the blunted heart rate (HR) dip during sleep and all-cause mortality in 3957 patients aged 55±16 (mean±SD) years (58% treated for hypertension) who were referred for ABPM between 1991 and 2005. The patients included dippers and non-dippers. HR measures during sleep, and, in particular, the absence of dipping of the HR to normal sleep levels, were independently associated with all-cause mortality (Ben-Dov et al., Arch Intern Med. 2007; 167: 2116-2121).

Thus, normalization of the non-dipper circadian BP and HR patterns (and the resulting RPP calculation of myocardial oxygen demand) to a dipper profile is a recently recognized therapeutic goal. Accumulating medical evidence suggests that such normalization may delay the progression towards the renal and cardiovascular pathology known to be associated with the non-dipper BP pattern.

Calcium channel blockers (CCBs) have now been used for decades in the treatment of cardiovascular diseases, such as hypertension and angina. CCBs can be divided into three distinct subgroups according to their chemical structures: the dihydropyridines (e.g., amlodipine), the phenylalkylamines (e.g., verapamil), and the non-dihydropyridine benzothiazepines (e.g., diltiazem).

The pharmacologic/clinical effects of all CCBs are similar, but there are also important differences between them. The dihydropyridines may cause renal adverse effects. For example, amlodipine is specifically not recommended for use in renally compromised patients and in certain populations at risk of developing renal disease. An important distinction between dihydropyridine CCBs, like amlodipine, and non-dihydropyridine CCBs, like diltiazem hydrochloride, is that the non-dihydropyridines do not interfere with autoregulation of the kidney and thus are less likely to lead to a high risk of proteinuria and eventually kidney disease and failure, to which hypertensives and especially certain segments of the hypertension population, such as African-Americans and diabetics, are particularly predisposed.

With respect to phenylalkylamines, verapamil, for example, has nearly three times the negative inotropic effects of diltiazem, which affects cardiac muscle contraction. Verapamil must be used with caution in compromised CV patients and verapamil has been associated with other side effects, such as constipation.

Diltiazems, such as Blue Note Pharmaceuticals' BNP 32,762 proprietary formulation, are non-dihydropyridine benzothiazepine CCBs. Diltiazem ((25-cis-3-(acetoxyl)-5-[2-(dimethylamino)ethyl]-2-3-dihydro-2-(4-methoxyphenyl)-1,5-benzothiazepin-4(5H)-one) has been demonstrated to be a safe and effective therapy for stable and unstable angina pectoris (Merck and Co., Inc. Diltiazem. Merck Index 1996, 12^{th} ed. No. 3247, p. 3249). Many formulations containing diltiazem or diltiazem hydrochloride are now available; they are used to provide a range of dose regimens, including dosing four times per day (“q.i.d.”), twice per day (“b.i.d.”), and once-daily (“q.d.”).

Patient compliance with dosing regimens was a factor in the development of the sustained or extended release formulations, enabling twice- or once-daily dosing. Cardizem CD is a once-a-day extended release formulation of diltiazem hydrochloride, for the treatment of hypertension and angina (Marion Merrill Dow. Cardizem CD Capsules. Physician's Desk Reference. 1995. 49^{th} ed. p. 1399). Dilacor XR is an extended release formulation of diltiazem hydrochloride; the capsules contain a controlled release formulation of diltiazem, intended to release the drug over a 24-h period, using the Geomatrix controlled release system (Rhone-Poulenc Rorer. Dilacor XR. Physician's Desk Reference. 1995. 49^{th} ed. p. 1966). Diltiazem OD ER (once-a-day, extended release, as described in the literature) is an extended release formulation, approved for the treatment of hypertension (Forest Pharmaceuticals, Inc. Tiazac. Physician's Desk Reference. 1998, 52^{nd} ed. pp. 957-959).

U.S. Pat. No. 4,721,619 (the “'619 patent”) described a controlled absorption diltiazem formulation, intended for twice daily usage. The formulation included a core containing diltiazem, and a multi-layer membrane surrounding the core. Water-soluble and -insoluble polymers used in the membrane, and the number of layers of membrane surrounding the core, affected the release rate of the drug from the core over a 12-h period. Peak plasma levels were obtained approximately 9-12 h after oral administration.

U.S. Pat. Nos. 4,894,240, 4,917,899, 5,002,776, and 5,616,345 described controlled absorption formulations for diltiazem, based upon the formulation described in the '619 patent. In U.S. Pat. No. 4,894,240, the formulation was designed for once-daily administration and provided maximal release of diltiazem ˜10-14 h after oral administration. The formulation described in U.S. Pat. No. 4,917,899 is characterized by its maximum release of diltiazem ˜9 h after administration. This formulation included a mixture of slow-release and fast-release pellets of diltiazem, the fast-release pellets having a thinner coating than the slow-release pellets.

U.S. Pat. No. 5,286,497 (the “'497 patent”), and U.S. Pat. Nos. 5,439,689 and 5,470,584 describe diltiazem formulations intended for once-daily administration. These formulations contain a mixture of two types of beads, described as a rapid-release bead and a delayed-release bead. According to the '497 patent, when tested separately, the rapid-release beads reached maximal release within 6-8 h after oral administration, while the delayed-release beads reached maximal release within 16-24 h post-administration. The beads contained a core, comprising diltiazem, and may contain conventional pharmaceutical excipients, while the coating was a polymer that enveloped or substantially enveloped the core, thereby effecting the controlled release characteristics of the drug from the core. According to the '497 patent, this formulation followed a “stair step” pattern of drug release, achieving a first maximum level of drug release at ˜6-8 h, and a second maximum after ˜21 h, in in vitro dissolution tests. When administered to humans, the formulation achieved a maximum plasma level at ˜6-8 h post-administration, with plasma levels dropping slowly over the next 10 h.

U.S. Pat. No. 5,508,040 described a multiparticulate pulsatile drug delivery system, containing a plurality of pellets or particles that were made up of two or more populations of pellets or particles. The particle populations each contained a core or bead containing the drug to be delivered, and a polymer film coating surrounding the core. The thickness of the coating surrounding the core controlled the rate of release of the drug into its environment, such as the stomach. The result was a pulsatile manner of drug delivery: the drug was released from a first population of pellets over a time period of ˜4.5 h after administration, its maximum release occurring at ˜3 h, and falling to baseline levels by ˜4.5 h. The second population of pellets started releasing drug at ˜3 h after administration, peaking at ˜6 h, and falling to baseline levels at ˜7.5 h, and in embodiments where a third population of pellets was contained in the formulation, the release of drug from the third population followed the same ˜4.5-h distribution pattern as the first two particle populations, but the start of drug release was delayed. When shown graphically, the curve shows peaks and valleys as a function of time after administration, corresponding to the rising and falling levels of the released drug.

Verapamil is a non-dihydropyridine CCB with a different mechanism of action from diltiazem or the dihydropyridine (amlodipine-like) CCBs. In the mid-1990s, Searle undertook development of an evening-dosed chronotherapeutic product to address the early morning surge in BP. After a number of promising early studies, a large outcome study was stopped two years early and the program was abandoned. A similar product, Cardizem-LA, was later fully developed and marketed.

The CONVINCE study was a double-blind, randomized comparative outcome study of chrono-verapamil versus a β-blocker and a diuretic. The study enrolled 16,602 people from 661 centers in 15 countries. All participants had hypertension and one or more risk factors for CVD. Follow-up was planned for five years but was stopped after a mean of three years by the sponsor (G D Searle, then Monsanto, Pharmacia) before the results were unblinded. The trial was stopped against the advice of the data safety monitoring board (DSMB) for commercial (transfer of ownership), not scientific reasons. Thus, no scientific conclusion can be drawn from the study.

Verapamil may not have been the optimal drug: diltiazem may be better suited to such a chronotherapeutic approach. There was a higher rate of heart failure with verapamil. Additionally, a single drug product is less attractive than a combination for broad-based and optimally effective 24-h coverage.

Diltiazem ((25-cis-3-(acetoxyl)-5-[2-(dimethylamino)ethyl]-2-3-dihydro-2-(4-methoxyph enyl)-1,5-benzothiazepin-4(5H)-one) (Merck and Co., Inc. Diltiazem. Merck Index 1996, 12th ed. No. 3247, p. 3249) is recognized as a safe and effective treatment for both stable and unstable angina pectoris. In terms of efficacy, diltiazem has been reported to be as effective as β-adrenergic receptor blockers (Cohn (1994) Clin. Cardiol. 17, 415-421; Chan et al. (1988) Am. Heart J. 116, 838-848; Humen et al. (1986) J. Am. Coll. Cardiol. 7, 329-335; Kenny et al. (1985) Br. Heart J. 53, 43-46; Kostuk & Pflugfelder (1987) Circulation 75, VI 14-121), nitrates (Cohn (1994) Clin. Cardiol. 17, 415-421; Chan et al. (1988) Am. Heart J. 116, 838-848) and verapamil (alpha-[3-[[2-(3,4-dimethoxyphenyl)ethyl]methylamino]propyl]-3,4[dimethoxy-alpha-(1-methylethyl)-benzeneacetonitrile (Merck & Co., Inc. Verapamil. Merck Index 1996 12th ed. No. 10083, p. 1696; Chaffman et al. (1985) Drugs 29, 387-454) and more effective than nifedipine (1,4-dihydro-2,6-dimethyl-4-(2-nitrophenyl)-3,5-pyrdinecarboxylic acid dimethyl ester) (Merck & Co., Inc. Nifedipine. Merck Index 1996 12th ed. No. 6617, p. 1121; Frishman et al. (1988) Circulation 77, 774-786).

Comparative studies have demonstrated approximately equivalent anti-anginal effects for many of the calcium channel blockers on the market. Such studies have also shown a lower incidence of side effects with diltiazem (e.g., Opie (1988) Cardiovasc. Drug Ther. 1, 464-491). Diltiazem is a widely prescribed medication, with large worldwide sales.

Metabolic studies have shown that diltiazem, like other calcium channel blockers, undergoes extensive first-pass metabolism in the liver, with a plasma half-life in the range of ˜2-6 h (Hoglund & Nilsson (1989) Ther. Drug Monit. 11, 543-550; Boyd et al. (1989) Clin. Pharmacol. Ther. 46, 408-419). Thus, a single oral dose of an immediate-release formulation of diltiazem must be administered three or four times per day to provide adequate control of hypertension or angina (Bala Subramanian et al. (1990) J. Am. Coll. Cardiol. 1, 1144-1153; Boman et al. (1995) Eur. J. Clin. Pharmacol. 49, 27-30; McCans (1986) Can. J. Cardiol. 2, 332-337.). Patient compliance with such a dosing regimen is often poor (Farmer et al. (1994) Clin. Ther. 16, 316-326). Additionally, many of the side effects associated with immediate release formulations of diltiazem likely result from the high peak plasma levels of drug that occur following the immediate release and before complete distribution.

To provide therapeutic blood levels of diltiazem for longer periods of times and to improve patient compliance, several slow-release or extended-release formulations of diltiazem have been developed for the treatment of hypertension and angina (e.g., Lacouriere et al. (1995) Am. J. Hypertension 8, 282-286; Guimont et al. (1993) Biopharm. Drug Dispos. 14, 767-778; Dupont et al. (1991) Cardiovasc. Drugs Ther. 5, 701-708) and angina (Cutler et al. (1995) J. Clin. Pharmacol. 35, 189-195; Thadani et al. (1994) Am. J. Cardiol. 74, 9-17; Weiner et al. (1986) Am. J. Cardiol. 57, 6-9; Khinke et al. (1995) Cardiovasc. Drugs Ther. 9, 319-330). Such formulations may provide adequate therapeutic blood levels during the patient's sleeping hours and upon waking in the morning, as well as improved patient compliance.

As used here, the extended release formulation of diltiazem hydrochloride, marketed as Cardizem CD, may be referred to as diltiazem CD, and as a slow or delayed release preparation of diltiazem. Dilacor XR, an extended-release formulation of diltiazem hydrochloride, may also be referred to as diltiazem ER (extended release), and as a slow or delayed release preparation of diltiazem. Diltiazem OD ER (once-a-day, extended release, as described in the literature) is an extended release formulation of diltiazem and is also referred to as a slow or delayed release preparation of diltiazem.

As described in U.S. Pat. No. 6,162, 463 (the “'463 patent”), an analysis of the anti-anginal effects of the three major once-daily, extended release formulations of diltiazem revealed that all three only produced partial reductions in angina. Diltiazem OD ER and diltiazem XR both maximally reduced the number of angina attacks by ˜50-60%, regardless of how high a dose was given. Both diltiazem OD ER and diltiazem XR reduced angina attacks at doses of 120 mg, but no further effects were seen at doses up to 540 mg. The situation for diltiazem CD was reported to be similar (Thadani et al. (1994) Am. J. Cardiol. 74, 9-17). These continuing residual angina attacks can be uncomfortable for the patients, and even potentially life-threatening.

Analysis of the chronobiology of angina attacks in relation to the pharmacokinetics of these drugs is useful in identifying the particular deficiency in the once-daily, extended release formulations of diltiazem. A diurnal rhythm for angina attacks has been reported, with ˜40-50% of these attacks occurring between 6:00 am and noon (Taylor et al. (1989) Am. Heart J. 118, 1098-1099; Mulcahy et al. (1988) Am J. Med. 81, 2-6). Taylor et al. (1989) reported that almost all attacks occurred during the waking hours, in two distinct phases (Am. Heart J. 118, 1098-1099). The first phase was in the morning, beginning at approximately 6:00 am and reaching a peak between approximately 8:00 am and 10:00 am. The second phase began at approximately 1:00 pm and lasted about 8 h. Other studies have shown similar diurnal rhythms for myocardial infarction (Muller et al. (1985) N. Engl. J. Med. 313, 1315-1322), ischemic ST segment depression (Rocco et al. (1987) Circulation 75, 395-400), and sudden cardiac death (Muller et al. (1987) Circulation 75, 131-138). These studies point to the importance of achieving appropriate medication levels during the pre-waking and morning hours, when a significant amount of abnormal cardiac activity occurs.

Extended-release formulations of diltiazem are generally taken in the morning. Diltiazem is slowly released from these formulations and slowly absorbed, so as to provide an extended and long-lasting dosage. Thus, by their nature, such once-daily extended release formulations of diltiazem are unable to provide sufficient levels of medication in the early morning, and especially in the pre-waking hours. Plasma levels of diltiazem OD ER, diltiazem CD (Cardizem CD) and Diltiazem XR (Dilacor XR) increase slowly and reach peak levels only after ˜4-6 h after ingestion. Thus, for drugs taken at, for example, about 8:00 am, peak plasma levels are not reached until between approximately 12:00 noon and 2:00 pm. This slow absorption of diltiazem is not altered much by increasing the dose. These data suggest that existing once-daily, extended-release formulations of diltiazem, if administered upon waking or at breakfast, are unable to provide sufficient plasma levels of drug when their presence is most necessary, in the early morning.

Example 1 of the '463 patent describes the preparation of a diltiazem formulation to address achieving appropriate medication levels during the morning hours, when a significant amount of abnormal cardiac activity occurs. Specifically, the contents of two 180 mg capsules of Dilacor XR were emptied, yielding 360 mg of diltiazem hydrochloride. This preparation was mixed with 120 mg of diltiazem hydrochloride. Dilacor XR is an extended release formulation of diltiazem hydrochloride, and is referred to here as a slow release (or delayed release) diltiazem preparation. Diltiazem hydrochloride is an immediate release formulation of diltiazem, referred to here as a quick release diltiazem preparation. Then, 120 mg of diltiazem hydrochloride powder was mixed with 360 mg of Dilacor XR, producing an extended release formulation of diltiazem hydrochloride.

Because various pharmaceutical excipients were used in the formulation of Dilacor XR, the weight of the contents of the capsule was greater than the weight of the drug contained therein. Thus, the weight percentages described are based upon the percent weight of the drug in the preparation (i.e., whether the quick release or the slow release preparation) in relation to the quantity of the active drug in the mixture. Thus, the mixture contained ˜25%, by weight, of a quick-release diltiazem preparation, and ˜75%, by weight, of a slow release diltiazem preparation, based on the percentage of diltiazem in the final mix.

This mix was then encapsulated in a gelatin capsule and administered orally to a healthy individual, taking no medications that would affect diltiazem plasma levels or interfere with their proper determination. Controls containing either 120 mg diltiazem hydrochloride or 360 mg diltiazem ER were administered separately. An interval of approximately 5-7 days elapsed between the administration of each agent tested in the example.

An intravenous blood sample was withdrawn, using ethylenediamine tetraacetic acid (EDTA) as an anticoagulant, just before administration of the test agent, representing the zero-time sample. At timed intervals after administration, additional blood samples were withdrawn and similarly treated. The blood samples were centrifuged for 15 min under conditions known to those skilled in the art. The plasma portion of each sample was harvested, frozen, and stored at −70° C. until analysis. The plasma levels of diltiazem were determined by high-performance liquid chromatography (HPLC) analysis (Eradiri & Midha 1995. Pharm. Res. 12, 2071-2074).

From these experiments, peak plasma levels of diltiazem were reached between ˜1-2 h after administration of the immediate-release formulation (quick-release preparation). Most of the quick-release preparation was gone by ˜5-6 h after administration. Diltiazem XR, the extended-release formulation (or slow-release preparation) individually, reached peak plasma levels at ˜6-7 h after administration, and maintained these peak levels for a further ˜10-12 h (FIG. 11).

The formulation of Example 1 of the '463 patent achieved peak plasma levels within 1-2 h after administration, and maintained those peak levels for ˜6 h, during which time plasma levels of individually administered diltiazem hydrochloride decreased and during which time the plasma level of individually administered diltiazem XR was increasing and reaching its peak plasma level. The plasma levels of the formulation of Example 1 of the '463 patent were sustained for ˜16 h before they began to decline (FIG. 11).

Most angina attacks have been shown to occur within the first ˜2 h after waking. The formulation in Example 1 of the '463 patent achieved maximal release of diltiazem within ˜2 h after oral administration, and maintained almost the peak diltiazem level for an extended time thereafter. Thus, as stated in the '463 patent, the invention could provide complete coverage, both in the morning and in the afternoon, to achieve suppression of angina attacks.

However, the formulation described in the '463 patent, with morning dosing, does not address the pre-waking and immediately post-waking rise in BP and HR.

Thus, there remains a continuing need for a chronotherapeutically designed, timed- and extended-release formulation of two or more pharmaceutically active compounds, or pharmaceutically active salts thereof, that allows once-daily morning dosing of a combination drug formulation, with timed morning and evening release characteristics, to achieve an effective plasma concentration of an anti-hypertensive drug in the pre-waking hours, to blunt the slope of the natural surge in BP and HR (and resulting myocardial oxygen demand, as reflected in the RPP) in the early morning, and to normalize or modify the non-dipper circadian BP pattern to a dipper profile, or a less severe non-dipper profile. Not least for patient compliance reasons, a once-daily formulation is desirable. Such combination drug formulations, with timed morning and evening release characteristics, may also exhibit synergistic effects, allowing for effective control of BP with reduced doses of one or more of the combination component drugs.

### BRIEF DESCRIPTION OF THE FIGURES

FIG. 1. Possible causes of morning increase in the incidence of coronary events. In diurnally active persons, acute cardiovascular events occur with increased frequency between the hours of 6:00 am and 12:00 pm, relative to other times of the day or night.

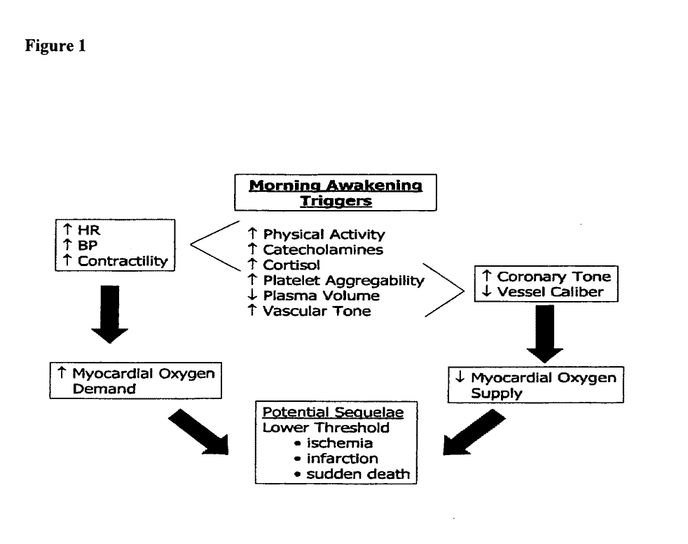

FIG. 2. Circadian pattern of systolic BP (SBP; left) and HR (right) in young, clinically healthy normotensive men and women, sampled by ABPM for 48 consecutive hours. Each graph shows the hourly means and standard errors (thin lines) of data collected from men (continuous line) and women (dashed line). Nonsinusoidal-shaped curves (thick lines) represented around means and standard errors correspond to the best-fitted waveform model, determined by population multiple-component analysis for each gender. Arrows descending from upper horizontal axis point to the circadian orthophase (rhythm crest time). The lower horizontal axis represents circadian time of sampling (in hours after awakening from nocturnal sleep). Average duration of sleep across all individuals is represented by the dark bar on the lower horizontal axis (modified from Hermida et al. (2002) Chronobiol. Int. 19; 461-481).

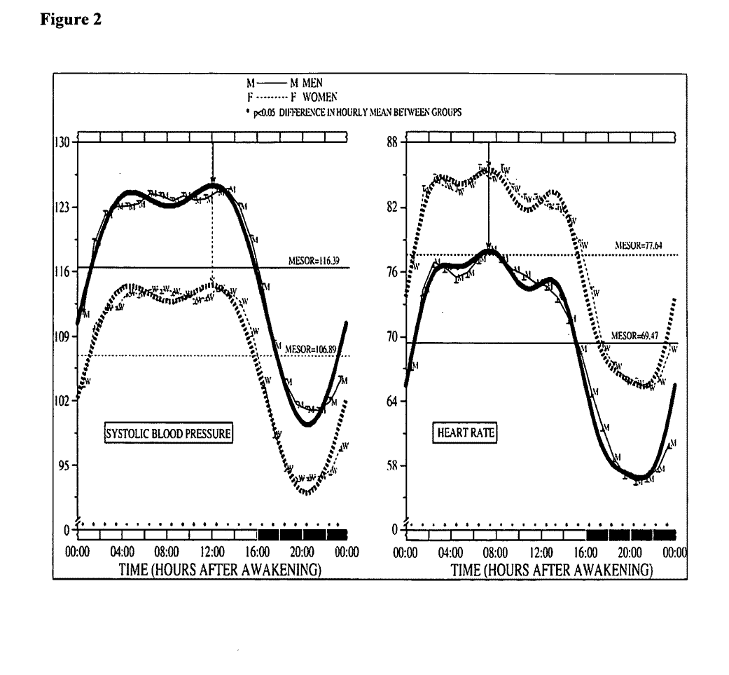

FIG. 3. The morning blood pressure surge.

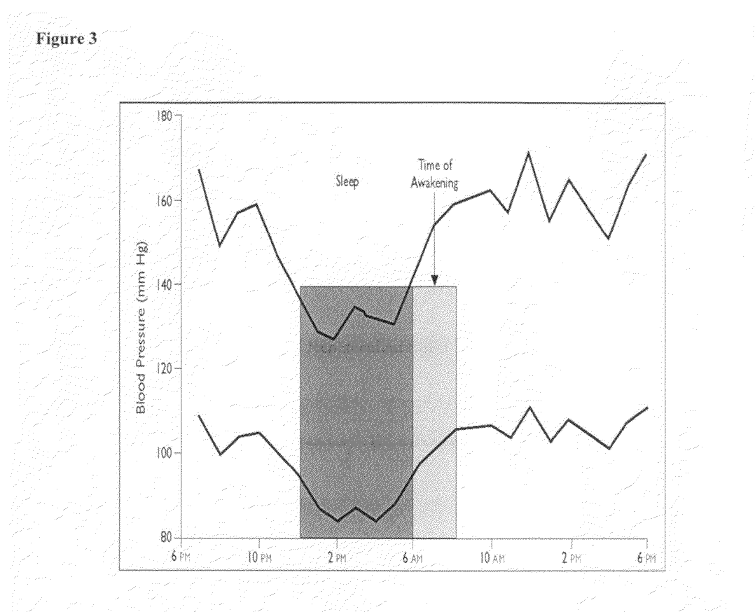

FIG. 4. Clock-like illustration of the organization of the circadian time structure shown by the peak time location of selected rhythms relative to the sleep (10:30 pm to 6:30 am) activity (6:30 am to 10:30 pm) routine of healthy persons. ACTH=adrenocorticotropic hormone; FSH follicle stimulating hormone; LH=luteinizing hormone; TSH=thyroid stimulating hormone; WBC=white blood cells (modified from Patient Care. Medical Economics. Smolensky MH, Bing ML: Chronobiology and chronotherapeutics in primary care. Patient Care Clin Focus 1997; 31 (summer suppl): 1-15).

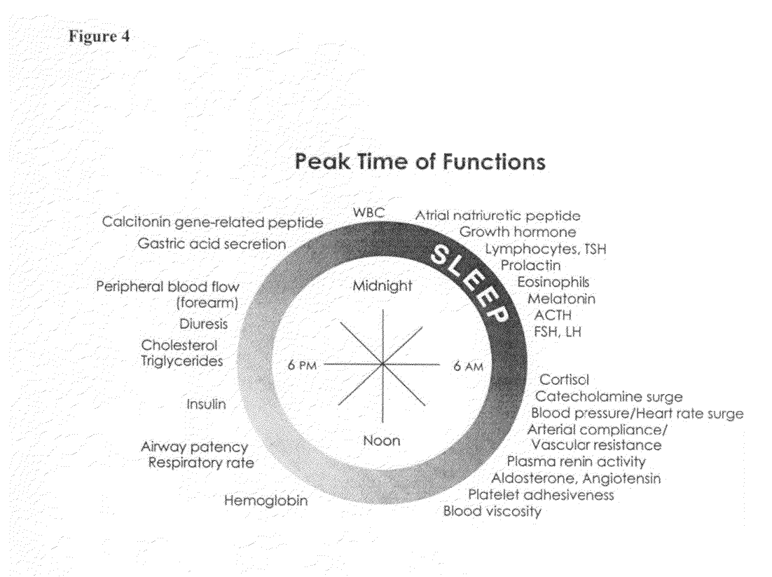

FIG. 5. Excess incidence of (A) stroke and (B) myocardial infarction (MI) in the peri-awakening period (from Clinical Cornerstone 2004; 6: 7-17)).

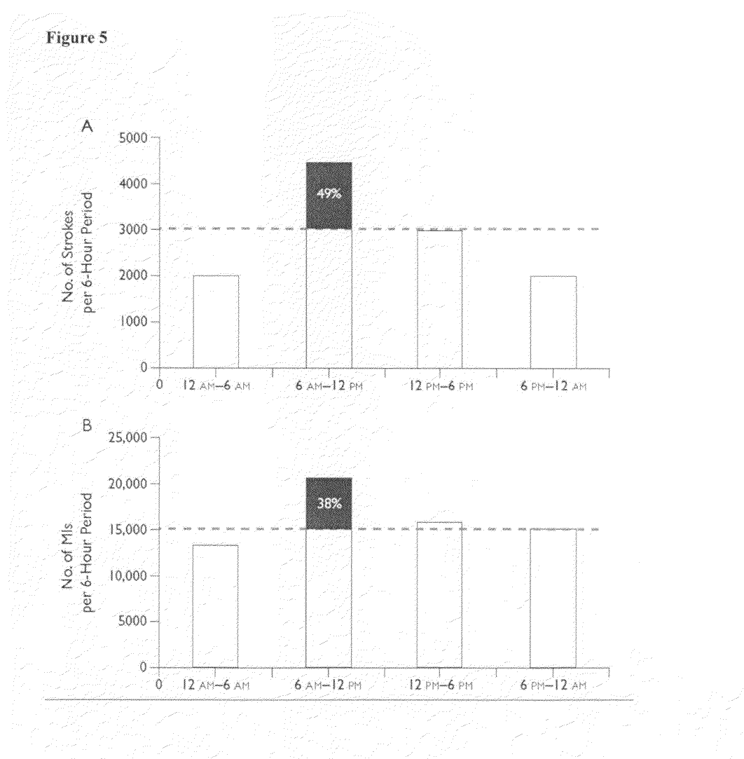

FIG. 6. Time and incidence of myocardial infarction in 1726 patients (from Willich et al. (1989) Circulation 80, 853-858).

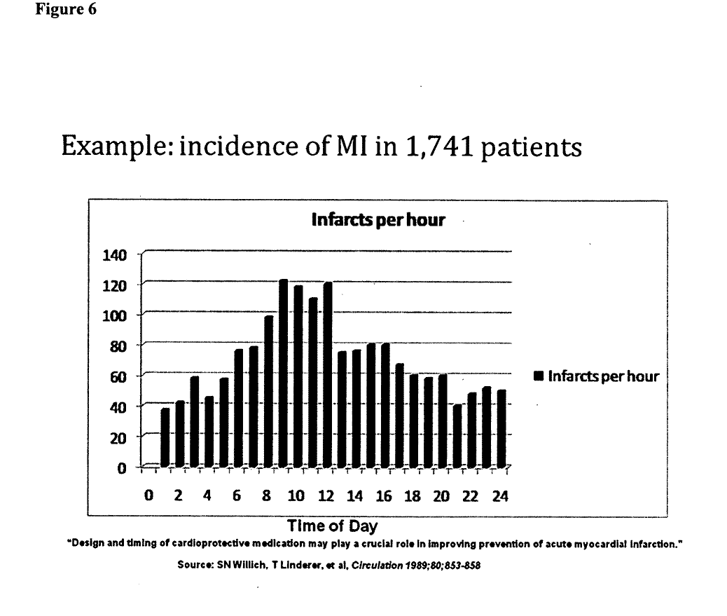

FIG. 7. Relationship between RPP and silent ischemic events in patients with silent coronary artery disease.

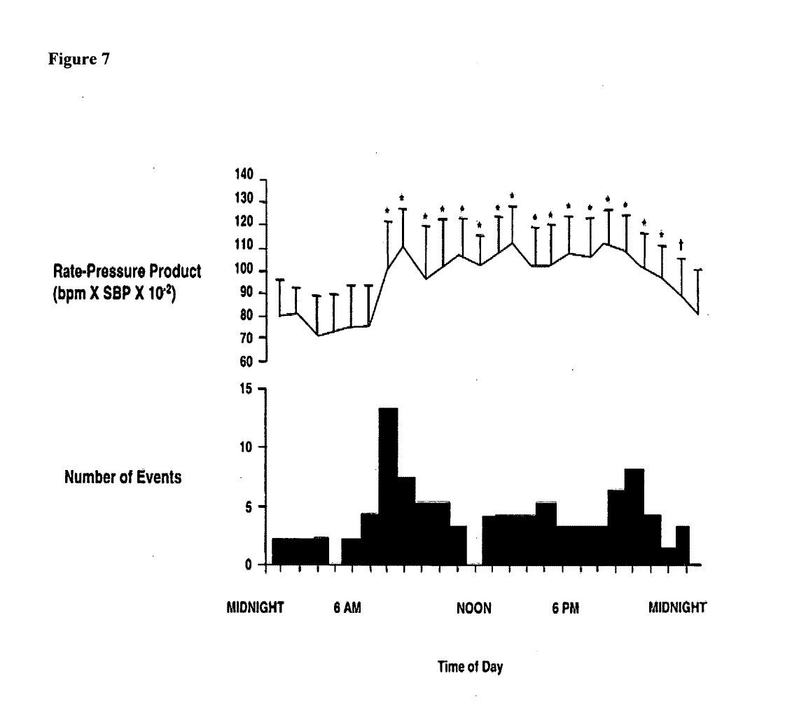

FIG. 8. Cox survival curves of the study population, subdivided according to dipping status. The model included age (exponential term), gender, treated diabetes, treated hypertension, and dipping as predictor variables and all-cause mortality as the outcome variable. The hazard ratios (95% confidence intervals) were 1.00 for subjects with a 10% or higher dip in both systolic blood pressure (SBP) and heart rate (reference group, n=1658); 1.39 (0.98-1.98) in patients with heart rate dip, but no SBP dip (n=756); 1.46 (1.05-2.04) in patients with SBP dip but no heart rate dip (n=774); and 1.90 (1.37-2.64) in patients with no dip (n=654) (from Ben-Dov et al. (2007) Arch Intern Med. 167: 2116-2121).

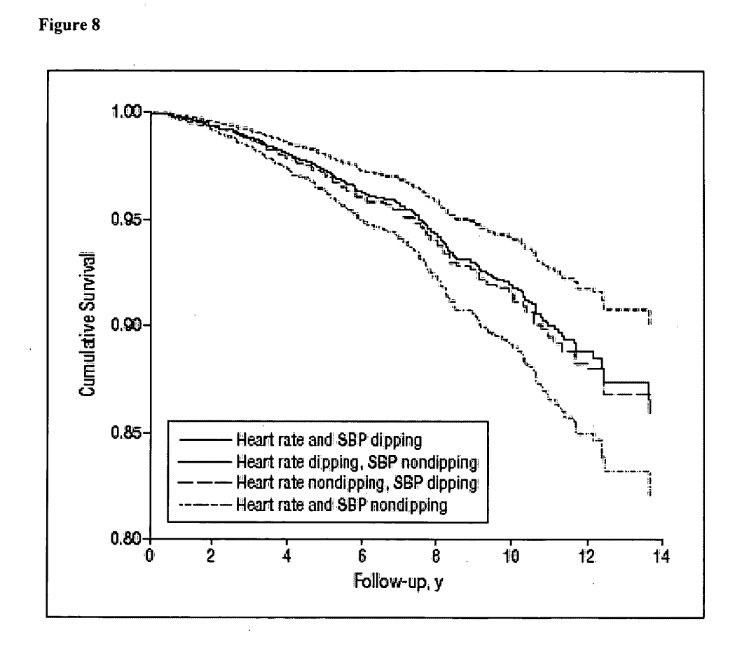

FIG. 9. Simulated combination product PK release profile.

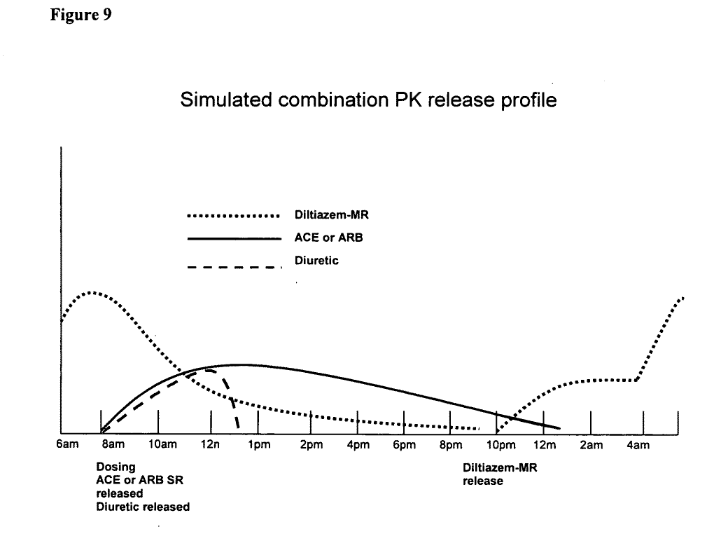

FIG. 10. In an embodiment of the present invention, a pill or bead layered formulation design is used. The outer shell immediately releases an ACE-I or ARB formulation (plus optionally, a diuretic, in a triple active ingredient product), with a slow release throughout the day. When this ACE-I or ARB has fully dissolved between ˜4 and ˜12 h later, a diltiazem CCB component in a weight ratio of ˜75% to ˜99% of delayed (extended) release (ER) and between ˜1% and ˜25% of immediate release (IR; such as 75% ER: 25% IR, 80% ER: 20% IR) is then released (i.e., a sustained-release diltiazem component first, followed by an immediate release bolus in the pre-waking hours (within ˜1-4 h before waking)).

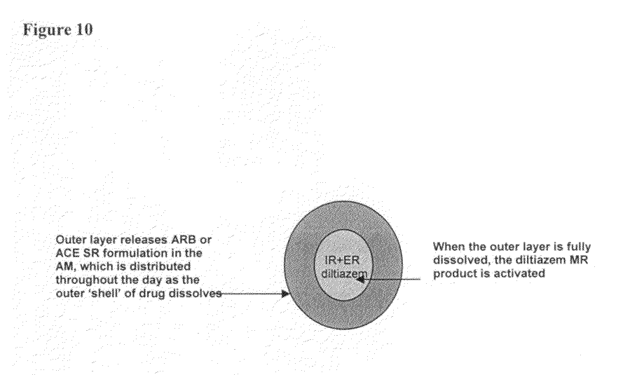

FIG. 11. In an embodiment of the present invention, a pill or bead layered formulation design/technology is used. The outer shell immediately releases a diltiazem component in a weight ratio of ˜1% to ˜25% of immediate release (IR) and between ˜75% and ˜99% of delayed (extended) release (ER; such as 25% IR: 75% ER, 20% IR: 80% ER), optionally with a diuretic in a triple drug product. When the diltiazem-MR is fully dissolved, the ACE I or ARB part of the formulation is then released, within ˜4 to ˜12 h post dosing.

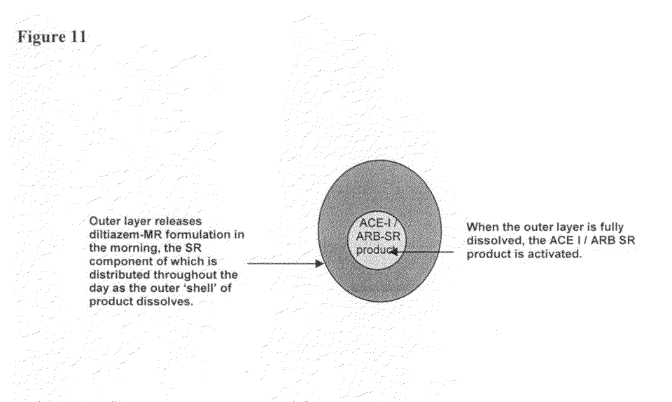

FIG. 12. The formulation of Example 1 of the '463 patent achieved peak plasma levels within about 1-2 h after administration, and maintained those peak levels for about 6 h. Example 1 of the '463 patent describes the preparation of a diltiazem formulation to address achieving appropriate medication levels during the morning hours, when a significant amount of abnormal cardiac activity occurs. The solid line refers to a preparation described in the '463 patent. The dotted line refers to the Tiazac product (a once-a-day, extended release product, as described in the literature, approved for the treatment of hypertension (Forest Pharmaceuticals, Inc. Tiazac. Physician's Desk Reference. 1998, 52 ed. pp. 957-959)).

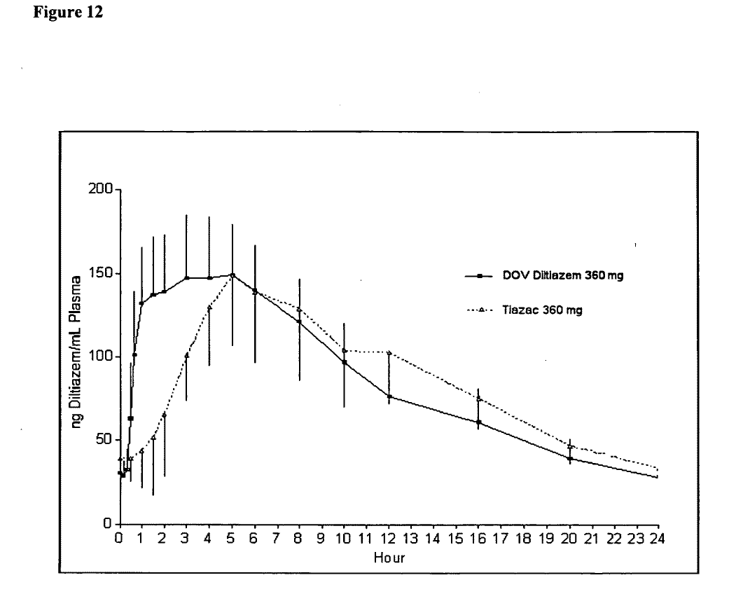

FIG. 13. Diltiazem modified release PK profile. In this example, the release and pharmacokinetic characteristics of the ARB component of the combination, shown separately in FIG. 14, would be initiated in the ˜4-12 h window on the diltiazem timescale.

FIG. 14. The release and pharmacokinetic characteristics of the ARB component of the combination (see Example 8, FIG. 13).

### DESCRIPTION OF THE INVENTION

The present invention comprises chronotherapeutically designed, timed- and extended-release formulations of two or more pharmaceutically active compounds, or pharmaceutically active salts thereof, that allow once-daily dosing of the combination drug formulation, with timed morning and evening release characteristics, to achieve an effective plasma concentration of an anti-hypertensive agent in the pre-waking hours, to blunt the slope of the natural surge in BP and HR (and resulting myocardial oxygen demand, as reflected in the RPP) in the early morning, and to normalize or modify the non-dipper circadian BP pattern to a dipper profile, or a less severe non-dipper profile.

It is an object of the present invention to provide a timed- and extended-release formulation of two or more pharmaceutically active compounds, or pharmaceutically active salts thereof, that allows once-daily morning dosing of a combination drug product with timed morning and evening release characteristics.

Embodiments of the present invention include (a) a combination of Blue Note Pharmaceuticals' proprietary modified-release formulation (BNP 32,762) with morning release, or a modified version of BNP 32,762 for evening release, or a dihydropyridine calcium channel blocker, such as amlodipine, plus an angiotensin converting enzyme (ACE) inhibitor (ACE-I) or angiotensin II receptor blocker (ARB) morning or evening release (“CORE-24 Double-Release [DR]”) and (b) a combination of BNP 32,762 morning release (or a modified version of BNP 32,762 for evening release) or amlodipine, plus an ACE-I or ARB morning or evening release with the addition of a morning-release diuretic (“CORE-24 Triple-Release [TR]”).

Preferred embodiments use ARBs that are potent and have long plasma half-lives and/or active metabolites, such as losartan, irbesartan, candesartan, valsartan, eprosartan, telmisartan, and olmesartan, as well as those ARBs having cardioprotective characteristics, such as valsartan, Other preferred embodiments use ACE-Is that are potent and have long plasma half-lives, such as benazepril and perindopril, and/or active metabolites, as well as those having cardioprotective characteristics, such as perindopril erbumine. In some embodiments of the invention, addition of a diuretic may be preferred or medically indicated in some anti-hypertensive patients.

Preferred ARBs have the following characteristics: chronotherapeutic suitability (e.g., those suitable for immediate release and those with prolonged half-lives), single-dose combination suitability (e.g., low or comparatively low dosing levels), and drug-to-drug compatibility when used in combination with other anti-hypertensive drugs, such as calcium channel blockers. Preferred embodiments may also use ARBs that have cardioprotective characteristics, which may prevent or decrease the incidence of myocardial infarction or stroke, or reduce the risk of cardiovascular mortality or non-fatal myocardial infarction.

Preferred ACE-Is have the following characteristics: chronotherapeutic suitability (e.g., those suitable for immediate release and those with prolonged half-lives), single-dose combination suitability (e.g., low or comparatively low dosing levels), and drug-to-drug compatibility when used in combination with other anti-hypertensive drugs, such as calcium channel blockers. Preferred embodiments also may use ACE-Is that have cardioprotective characteristics, which may prevent or decrease the incidence of myocardial infarction or stroke, or reduce the risk of cardiovascular mortality or non-fatal myocardial infarction.

The diltiazem formulations of the present invention address the morning surges in BP and HR (and the rate-pressure product (systolic blood pressure×heart rate/100, RPP)), provide overnight BP and HR (and RPP) control in non-dippers, modulate the slopes of the morning BP and HR surges in both non-dippers and dippers, and address outcome potentials for target organ damage and CV events.

Advantages of using diltiazem in chronotherapy include that the effects of diltiazem in lowering heart rate may be of clinical relevance, particularly in the overnight hours for non-dippers and in the early morning/pre-waking hours for dippers and non-dippers. The effect of non-dihydropyridines, such as diltiazem, in slowing heart rate may provide an important distinguishing mechanism well suited to addressing circadian variability and CV outcomes. Benzothiazepine non-dihydropyridine CCBs are better suited than phenylalkylamines, such as verapamil, because of the latter's negative inotropic side effects. Rapidly acting dihydropyridine CCBs, such as amlodipine, may actually aggravate myocardial ischemia by causing reflex tachycardia. The use of non-dihydropyridines (diltiazem) represents a preferred embodiment of the invention in situations where patients have experienced or are at high risk of experiencing cardiovascular events, such as myocardial infarction, or in patients who have developed renal disease or are at risk of developing renal disease; however, other embodiments of the invention may include dihydropyridine CCBs, such as amlodipine besylate.

All embodiments of the present invention overcome the disadvantages of the presently available anti-hypertensive drug formulations. A diltiazem-MR or standard dose amlodipine composition of the present invention addresses circadian variation in BP patterns, because this variation is associated with important clinical events. The pre- and post-awakening surge in morning BP has been implicated as a potential factor contributing to the morning excess of serious cardiovascular events, such as MI and strokes. Lowering the rising BP during these key morning hours is thus a desirable feature in anti-hypertensive therapy. In combination with other anti-hypertensive agents, where dosing is linked to key times, the present invention provides desirable 24-h control of BP and affects CV outcomes tied to circadian rhythms.

Several studies have shown that resistant blood pressure is common, even in patients being treated with one or more anti-hypertensive drugs. The JNC-7 (“The Seventh Report of the Joint National Committee on Prevention, Detection, Evaluation, and Treatment of High Blood Pressure”) defined “resistant hypertension” as: “the failure to achieve goal BP in patients who are adhering to full doses of an appropriate regimen of at least three anti-hypertensive drugs that includes a diuretic.” Indeed, as many as 50-75% of people being treated for hypertension fail to meet their target blood pressure levels. In one trial, ˜50% of the participants had uncontrolled blood pressure levels when enrolled, despite already being treated for hypertension; ˜59% of them were being treated with a single drug, and ˜41% with two drugs. During the trial, ˜72% of patients needed more than one drug to reduce their diastolic blood pressures to 80 mmHg. In another trial in hypertensive diabetics, ˜60% of participants needed two or more drugs to achieve blood pressure levels of less than 150/85 mmHg, and ˜33% needed three or more drugs. A review by Sarafidis & Bakris (J. Clin. Hypertension 10, 130-139), entitled, “State of hypertension management in the United States: confluence of risk factors and the prevalence of resistant hypertension,” cites and explores the failure to adequately control BP, even in medicated patients at alarming percentages and the need to find better ways to provide broad and effective 24-h coverage.

Formulations of the present invention address the pre-waking/morning surges in BP and HR (and RPP), and provide better blood pressure control than existing formulations. The present invention comprises chronotherapy with anti-hypertensive drugs to align peak plasma drug levels with times when BP, HR, and myocardial oxygen demands are at highest levels, so as to refine 24-h management and beneficially affect CV outcomes. The once-daily drug products of the present invention, providing for the chronotherapeutic delivery of combinations of anti-hypertensive drugs provide more effective 24-h coverage, benefitting both dippers and non-dippers in the important overnight, pre-waking, and immediate post-waking hours, thus positively affecting therapy-resistant hypertension.

### EXAMPLES

The terms “agent,” “drug,” “pharmaceutical,” or “pharmaceutically active compound,” are used interchangeably when referring to a compound being released from a formulation.

As described in the literature, extended-release formulations of diltiazem or diltiazem hydrochloride contain diltiazem along with various pharmaceutical excipients, such as binders or other inert ingredients, on a pharmaceutically acceptable inert core or seed. Any of the binding agents known to those skilled in the art can be used, such as starch or other sugars. Other pharmaceutical excipients that may be used in formulating a slow release preparation include talc, stearates, acidifying agents, where necessary, and preservatives, such as antimicrobial compounds. These excipients may further include a lubricant, such as waxes, castor oil, and mineral oil.

The coating agents used to form the slow release preparation can be selected from a variety of compounds known to those skilled in the art. For example, the formulation in U.S. Pat. No. 5,286,497 uses a polymeric coating of acrylate compounds, though they describe other compounds that can be used. The formulation described in U.S. Pat. No. 4,917,899 similarly used a coating prepared from acrylate polymers to effect the slow release of diltiazem from the core. Methods of encapsulating or tableting these preparations are also known to those skilled in the art. Hard or soft gelatin capsules may be used.

All references cited in the specification are hereby incorporated by reference, to the extent pertinent.

### Example 1

An embodiment of the present invention comprises a novel formulation of modified-release (MR) diltiazem hydrochloride, in a weight ratio of ˜75% to ˜99% of delayed (extended) release (ER) and between ˜1% an ˜25% of immediate release (IR) (such as 75% ER:25% IR, 80% ER:20% IR), with a release profile, such that the ER formulation releases on dosing and an IR formulation releases within 4-12 h later.

This embodiment is suitable for use as a stand-alone HS (bedtime) dosing product or for use in a combination product, with one or more other anti-hypertensive drugs, to achieve the key clinical objectives of the product, whereby the other drugs would release in the morning and the new MR-diltiazem formulation would release in the evening, thus ensuring 24-h control of BP and the preferred diltiazem effect on BP and HR in the sleeping, pre-waking, and post-waking hours.

As an example, the release profile of a single-dose, double-combination product, for morning dosing would provide an angiotensin II receptor blocker (ARB) or angiotensin converting enzyme (ACE) inhibitor (ACE-I) component that is released immediately. As another example, an ARB or ACE-I component is released immediately, along with a diuretic.

The modified release (MR) diltiazem component is released at 4-12 hours post-dosing, providing sustained release (SR) diltiazem coverage overnight, with an extra bolus of immediate release (IR) drug to occur in the pre-waking hours (e.g., in the period 1-4 h before waking)). Thus, ˜75% or more of SR drug is released first, followed by a time-delayed release of an IR bolus of up to 25% later.

The diltiazem SR component provides overnight control of BP by exerting an anti-hypertensive effect, even in non-dippers. A lower net effect is expected for dippers, but one that, combined with the effect of the later IR component, advantageously blunts the slopes of the morning surges in HR and BP. The diltiazem IR component provides a pre-waking bolus release, providing anti-hypertensive coverage during the morning surge period (BP, HR, RPP) that helps to blunt the pre-waking surges in both non-dippers and dippers.

### Example 2

Another embodiment of the present invention comprises a formulation in which in the morning, there is release of a diltiazem component in a weight ratio of ˜1% to ˜25% of immediate release (IR) and between ˜75% and ˜99% of delayed (extended) release (ER) (such as 25% IR: 75% ER, 20% IR: 80% ER), reducing BP and HR during the important morning surge period, with therapeutic plasma levels achieved within ˜15-30 min, and providing sustained release thereafter of diltiazem, and optionally of a diuretic in a triple-combination product. Then, the ARB or ACE-I component is released within 4-12 hours later, providing overnight control of BP in two ways. First, it exerts an anti-hypertensive effect in non-dippers; there will be a lower net effect in dippers. In both dipper and non-dippers, the ARB or ACE-I will blunt the slope of the morning rise in BP and HR in the pre-waking hours, and there is expected to be a synergistic effect on post-waking BP and the slope of the rise in BP in the post-waking period, when coupled with the release of MR-diltiazem with the next morning dose.

### Example 3

An embodiment of the present invention comprises a formulation for morning dosing. The release profile comprises the immediate release (IR) of an ACE-I or ARB in the morning, followed by a modified release (MR) of diltiazem comprising ˜75% to ˜99% ER, starting within 4-12 h after the IR dose, plus up to ˜25% IR of further diltiazem in the period 1-4 h pre-waking.

In an embodiment of the invention, an enteric coating is used, comprising polymers that only dissolve at a pH of ˜6 or above. Such coatings can be applied to beads and tablets (including a bilayer or tablet within a tablet).

In another embodiment of the invention, an enteric coated capsule with beads is used. In another embodiment, a tablet is placed within a capsule. The enteric-coated capsule is filled with pH-independent beads which then release when the enteric-coated capsule dissolves at a high pH.

### Example 4

An embodiment of the present invention comprises a formulation for morning dosing. The release profile comprises the immediate release (IR) of an ACE-I or ARB in the morning, followed by a modified release (MR) of diltiazem or a standard dose of amlodipine, starting within 4-12 h after dosing.

In an embodiment of the invention, an enteric coating is used, comprising polymers that only dissolve at a pH of ˜6 or above. Such coatings can be applied to beads and tablets (including a bilayer or tablet within a tablet).

In another embodiment of the invention, an enteric coated capsule with beads is used. In another embodiment, a tablet is placed within a capsule. The enteric-coated capsule is filled with pH-independent beads, which then release when the enteric-coated capsule dissolves at a high pH.

### Example 5

An embodiment of the present invention comprises a formulation for morning dosing. The release profile comprises the immediate release (IR) of an ACE-I or ARB or diltiazem or amlodipine in the morning, and optionally a diuretic, followed by a modified release (MR) of diltiazem or standard release of amlodipine, starting within 4-12 h after dosing.

In an embodiment of the invention, an enteric coating is used, comprising polymers that only dissolve at a pH of ˜6 or above. Such coatings can be applied to beads and tablets (including a bilayer or tablet within a tablet).

In another embodiment of the invention, an enteric coated capsule with beads is used. In another embodiment, a tablet is placed within a capsule. The enteric-coated capsule is filled with pH-independent beads which then release when the enteric-coated capsule dissolves at a high pH.

### Example 6

An embodiment of the present invention comprises a formulation for morning dosing. The release profile comprises the IR of diltiazem or standard dose of amlodipine and a diuretic in the morning, followed by an extended release (ER) formulation of diltiazem (starting in the morning, with a ˜12 h release) or a standard dose of amlodipine, followed by the release of an ACE-I or ARB within 4-12 h after the IR diltiazem or standard dose amlodipine.

In an embodiment of the invention, an enteric coating is used, comprising polymers that only dissolve at a pH of ˜6 or above. Such coatings can be applied to beads and tablets (including a bilayer or tablet within a tablet).

In an embodiment of the invention, the formulation of Example 1 of the '463 patent is modified such that the dissolution rate is reduced to ˜12 h or less from ˜24 h.

In an embodiment of the invention, ACE-I or ARB core beads or a tablet are coated with ER diltiazem or amlodipine and IR diltiazem or amlodipine and a diuretic.

### Example 7

An embodiment of the present invention comprises a formulation for morning dosing. The release profile comprises the IR of diltiazem or standard dose amlodipine and a diuretic in the morning, followed in the case of the diltiazem embodiment by an extended release (ER) formulation of diltiazem, within ˜4-12 h after dosing.

In an embodiment of the invention, an enteric coating is used, comprising polymers that only dissolve at a pH of ˜6 or above. Such coatings can be applied to beads and tablets (including a bilayer or tablet within a tablet).

In an embodiment of the invention, the formulation of Example 1 of the '463 patent is modified such that the dissolution rate is reduced to ˜12 h or less from ˜24 h.

In an embodiment of the invention, ACE-I or ARB core beads or a tablet are coated with ER diltiazem or standard dose amlodipine and IR diltiazem or standard-dose amlodipine and a diuretic.

### Example 8

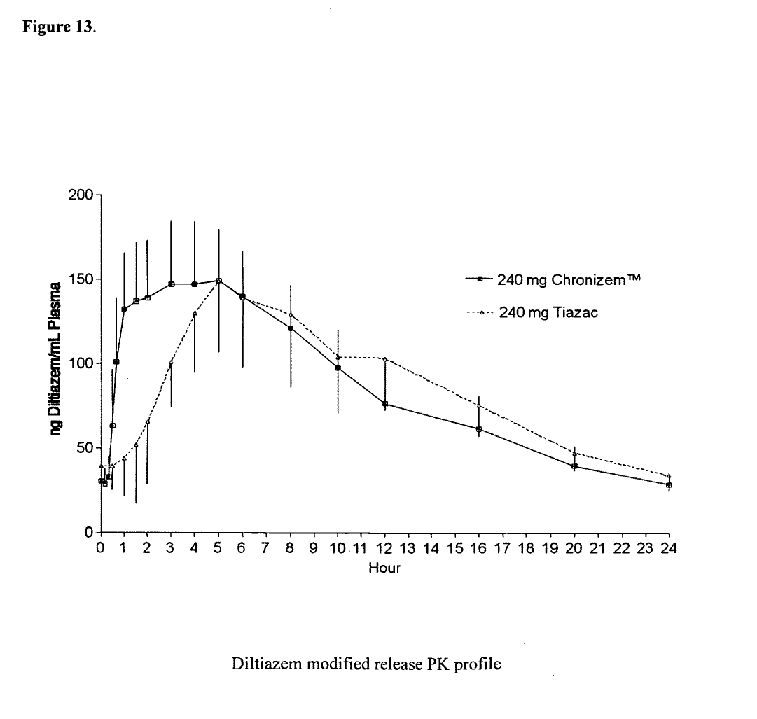

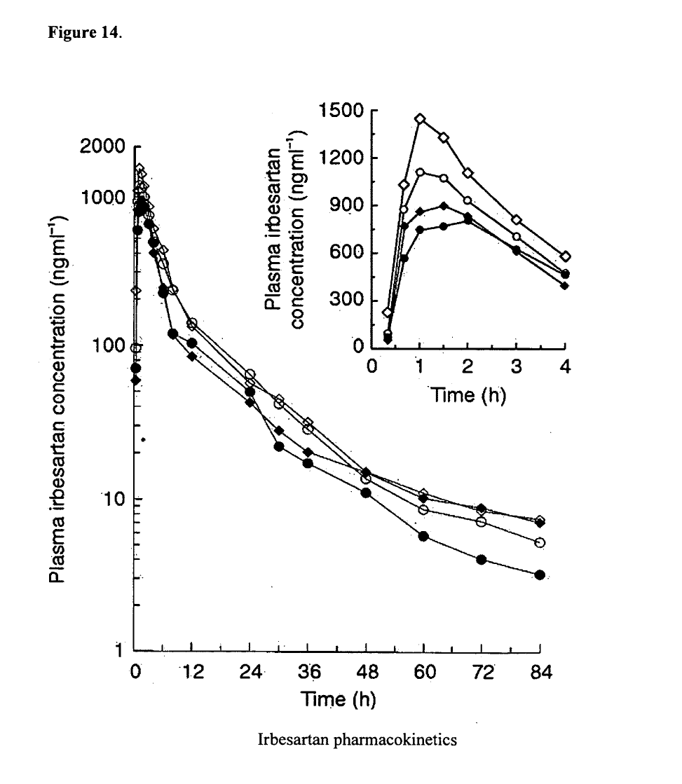

A composition for evening dosing comprising diltiazem, providing release on dosing of diltiazem, followed by release of an ARB (e.g., irbesartan) within about 4-12 h later. In this example, the release and pharmacokinetic characteristics of the ARB component of the combination (FIG. 14), would be initiated in the ˜4-12 h window on the diltiazem timescale (FIG. 13).

### Example 9

### Embodiments of the Present Invention Include

A composition for morning dosing, comprising an ACE-I or ARB, providing release of the ACE-I or ARB on dosing, followed within 4-12 h later by release of a diltiazem component in a weight ratio of ˜75% to ˜99% of delayed (extended) release (ER) and between ˜1% and ˜25% of immediate release (IR) (such as 75% ER: 25% IR, 80% ER: 20% IR), providing sustained release of diltiazem to provide overnight control of BP and HR, followed by a pre-waking bolus of IR diltiazem (within ˜1-4 h before waking), or alternatively substituting a dihydropyridine CCB such as a standard dose of amlodipine for the diltiazem component.
A composition for morning dosing, comprising an ACE-I or ARB or diltiazem or amlodipine, providing release of the ACE-I or ARB or diltiazem or amlodipine on dosing, followed within 4-12 h later by release of a diltiazem or amlodipine component, providing sustained release of diltiazem or amlodipine to provide overnight control of BP and HR, followed by, in the case of the diltiazem based embodiment, a pre-waking bolus of IR diltiazem (within ˜1-4 h before waking).
A composition for morning dosing, comprising an ACE-I or ARB or diltiazem or amlodipine, providing release of the ACE-I or ARB or diltiazem or amlodipine on dosing, followed within 4-12 h later by release of an ACE-I or ARB or diltiazem or amlodipine component, providing overnight control of BP and HR, and reducing the pre-waking/morning (within ˜1-4 h before waking) surge in BP and HR.
A composition for morning dosing, comprising an ACE-I or ARB or diltiazem or amlodipine, providing release of the ACE-I or ARB or diltiazem or amlodipine on dosing, followed within 4-12 h later by release of a diltiazem or amlodipine component that provides overnight control of BP and HR, followed by, in the case of the diltiazem-based embodiment, a pre-waking bolus of IR diltiazem (within ˜1-4 h before waking).
A composition for morning dosing comprising an ACE-I or ARB or diltiazem or amlodipine, providing release of the ACE-I or ARB or diltiazem or amlodipine on dosing, followed within 4-12 h later by, in the case of the diltiazem-based embodiment, release of a diltiazem component in a weight ratio of ˜75% to ˜99% of delayed (extended) release (ER) and between ˜1% and ˜25% of immediate release (IR; such as 75% ER: 25% IR, 80% ER: 20% IR), providing sustained release of diltiazem to provide overnight control of BP and HR, followed by a pre-waking bolus of IR diltiazem (within ˜1-4 h before waking).
A composition for morning dosing comprising an ACE-I or ARB and a diuretic, providing release of the ACE-I or ARB and the diuretic on dosing, followed within 4-12 h later by release of a standard dose of amlodipine or a diltiazem component in a weight ratio of ˜75% to ˜99% of delayed (extended) release (ER) and between ˜1% and ˜25% of immediate release (IR; such as 75% ER: 25% IR, 80% ER: 20% IR), providing sustained release of diltiazem to provide overnight control of BP and HR, followed by a pre-waking bolus of diltiazem (within ˜1-4 h before waking).
A composition for morning dosing comprising an ACE-I or ARB or diltiazem or amlodipine and a diuretic, providing release of the ACE-I or ARB or IR diltiazem or standard dose amlodipine and the diuretic on dosing, followed within 4-12 h later by, in the case of the diltiazem-based embodiment, release of a diltiazem component, providing sustained release of diltiazem to provide overnight control of BP and HR, followed by a pre-waking bolus of diltiazem (within ˜1-4 h before waking).
A composition for morning dosing comprising an ACE-I or ARB or diltiazem or amlodipine and a diuretic, providing release of the ACE-I or ARB and the diuretic on dosing, followed within 4-12 h later by release of a standard dose of amlodipine or a diltiazem component in a weight ratio of ˜75% to ˜99% of delayed (extended) release (ER) and between ˜1% and ˜25% of immediate release (IR; such as 75% ER: 25% IR, 80% ER: 20% IR), providing sustained release of diltiazem to provide overnight control of BP and HR, followed by a pre-waking bolus of diltiazem (within ˜1-4 h before waking).
A composition for morning dosing comprising diltiazem or amlodipine, providing on dosing either a standard dose of amlodipine or release of a diltiazem component in a weight ratio of ˜1% to ˜25% of immediate release (IR) and between ˜75% and ˜99% of delayed (extended) release (ER; such as 25% IR:75% ER, 20% IR:80% ER), that reduces BP and HR during the key morning surge period within ˜15-30 min after administration, and provides sustained release thereafter of diltiazem, followed by release of an ARB component within ˜4-12 h later, to provide overnight control of BP.
A composition for morning dosing comprising diltiazem or amlodipine, providing on dosing release of either a standard dose of amlodipine or release of a diltiazem component in a weight ratio of ˜1% to ˜25% of immediate release (IR) and between ˜75% and ˜99% of delayed (extended) release (ER; such as 25% IR:75% ER, 20% IR:80% ER) and a diuretic, that reduces BP and HR during the key morning surge period, within ˜15-30 min after administration, that provides sustained release thereafter of diltiazem, followed by release of an ARB component within ˜4-12 h later, providing overnight control of BP.

A composition for morning dosing comprising diltiazem or amlodipine, providing on dosing release of either a standard dose of amlodipine or a diltiazem component in a weight ratio of ˜1% to ˜25% of immediate release (IR) and between ˜75% and ˜99% of delayed (extended) release (ER; such as 25% IR:75% ER, 20% IR:80% ER), reducing BP and HR during the key morning surge period, within ˜15-30 min after administration, that provides sustained release thereafter of diltiazem, followed by release of an ACE I component within ˜4-12 h later, providing overnight control of BP.
A composition for morning dosing comprising diltiazem or amlodipine, providing on dosing release of either a standard dose of amlodipine or a diltiazem component in a weight ratio of ˜1% to ˜25% of immediate release (IR) and between ˜75% and ˜99% of delayed (extended) release (ER; such as 25% IR:75% ER, 20% IR:80% ER) and a diuretic, reducing BP and HR during the key morning surge period, within ˜15-30 min, and providing sustained release thereafter of diltiazem, followed by release of an ACE-I component within ˜4-12 h later, providing overnight control of BP.
A composition for evening dosing comprising diltiazem or amlodipine, providing on dosing either a standard dose of amlodipine followed by the pre-waking release of diltiazem or a diltiazem component in a weight ratio of ˜75% to ˜99% of delayed (extended) release (ER) and between ˜1% and ˜25% of immediate release (IR; such as 75% ER: 25% IR, 80% ER: 20% IR), with a release profile, such that the ER formulation releases on dosing and an IR formulation releases about ˜6 h later.
A composition for evening dosing comprising diltiazem or amlodipine, providing on dosing either a standard dose of amlodipine followed by the pre-waking release of diltiazem or a diltiazem component in a weight ratio of ˜75% to ˜99% of delayed (extended) release (ER) and between ˜1% and ˜25% of immediate release (IR; such as 75% ER: 25% IR, 80% ER: 20% IR), with a release profile, such that the ER formulation releases on dosing and an IR formulation releases within ˜4-12 h later.
A composition for evening dosing comprising diltiazem or amlodipine, providing on dosing either a standard dose of amlodipine followed by release of an ACE-I or ARB within 4-12 h later or a diltiazem component in a weight ratio of ˜75% to ˜99% of delayed (extended) release (ER) and between ˜1% and ˜25% of immediate release (IR; such as 75% ER:25% IR, 80% ER:20% IR), with a release profile, such that the ER formulation releases on dosing and an IR formulation releases ˜6-8 h later and an ACE-I or ARB releases within ˜4-12 h later.
A composition for evening dosing comprising diltiazem or amlodipine, providing on dosing either a standard dose of amlodipine followed by release of an ACE-I or ARB within 4-12 h later or a diltiazem component in a weight ratio of ˜75% to ˜99% of delayed (extended) release (ER) and between ˜1% and ˜25% of immediate release (IR; such as 75% ER:25% IR, 80% ER:20% IR), with a release profile, such that the ER formulation releases on dosing and an IR formulation releases ˜6-8 h later and an ACE-I or ARB releases within ˜4-12 h later.

### Example 10

In assessing the release profiles of the formulations of the present invention, in vitro procedures known to those of skill in the art are useful. In an example embodiment of the present invention,
at time zero to 1 h post-dose, there would be immediate release of diltiazem and optionally, a diuretic, such as hydrochlorothiazide,
at time ˜1 to ˜12 h post-dose, there would be release of an ER diltiazem component, and
at time ˜8 to ˜12 h post-dose, there would be a delayed release of irbesartan.

Standard dissolution methods are known to those of skill in the art and are available, for example, from the US FDA on their website. For example, the irbesartan/hydrochlorothiazide combination can be assessed with an in vitro dissolution method that uses apparatus II (paddle) in 0.1 N HCl medium at a rotation speed of 50 RPM. Irbesartan has low water solubility at low pHs (0.1 N HCl). However, in this example embodiment, it would be released later, when the pH value would be higher and thus irbesartan's solubility would be higher. For the diltiazem component, a suitable dissolution method is apparatus I (basket) at 100 RPM in aqueous medium.

With the skilled artisan's knowledge of available dissolution methods for the individual components of the combination formulation of the present example, the following 24-h two-stage dissolution method would be appropriate:
Stage 1 (acid): Apparatus I, 100 RPM in 900 mL of 0.1 N HCl for 2 h, switching to Stage 2 by replacing the medium, and
Stage 2 (buffer): Apparatus I, 100 RPM in 900 mL pH 7.5 phosphate buffer for the remaining 22 h.

Such a two-stage dissolution procedure, switching the medium, is a common approach when evaluating delayed release formulations. It is appropriate here because the release of the irbesartan is delayed and it would be helpful to demonstrate that the mechanism responsible for the delayed release (e.g., a film coating) is not influenced by the acidic environment of the stomach. The phosphate buffer at pH 7.5 approximately simulates the average pH in the colon. A similar dissolution method has been used to assess delayed-release mesalamine formulations, intended to provide colonic delivery.

The formulations and applications of the present invention are not intended to be limited to the embodiments illustrated in the examples, but only to the extent described in the following claims.

What is claimed is:

1. An anti-hypertensive composition for morning dosing, comprising a first anti-hypertensive agent, and optionally a diuretic, providing release of said first anti-hypertensive agent, and of said optional diuretic, on dosing, followed, within 4-12 h later, by release of a second anti-hypertensive agent.

2. The composition of claim 1 wherein said first anti-hypertensive agent is an ACE-I or an ARB or diltiazem or amlodipine, said second anti-hypertensive agent is diltiazem or amlodipine, and said optional diuretic is a thiazide diuretic or a thiazide-like diuretic.

3. The composition of claim 2 wherein said thiazide diuretic is selected from the group consisting of hydrochlorothiazide and chlorthalidone.

4. The composition of claim 2 wherein said thiazide-like diuretic is selected from the group consisting of a sulfonamide, indapamide, and metolazone.

5. The composition of claim 2 wherein the ACE-I is selected from the group consisting of benazepril, lisinopril, enalapril, perindopril erbumine, and ramapril.

6. The composition of claim 2 wherein the ARB is selected from the group consisting of losartan, irbesartan, candesartan, valsartan, eprosartan, telmisartan, and olmesartan.

7. The composition of claim 1 wherein said first anti-hypertensive agent is diltiazem, said second anti-hypertensive agent is olmesartan or irbesartan, and said optional diuretic is hydrochlorothiazide.

8. The composition of claim 2 wherein said second anti-hypertensive agent is in a delayed (extended) release (ER) form and an immediate release (IR) form, providing sustained release of said second anti-hypertensive agent, followed by a pre-waking bolus of said second anti-hypertensive agent.

9. The composition of claim 8 wherein release of said pre-waking bolus of said second anti-hypertensive agent occurs within about 1-4 hours before waking.

10. The composition of claim 8 wherein the weight ratio of said second anti-hypertensive agent is ˜75% to ˜99% of delayed (extended) release (ER) and between ˜1% and ˜25% of immediate release (IR).

11. The composition of claim 2 wherein the weight ratio of said first anti-hypertensive agent is ˜75% to ˜99% of delayed (extended) release (ER) and between ˜1% and ˜25% of immediate release (IR).

12. The composition of claim 11 wherein the release of said first anti-hypertensive agent begins within about 15-30 min after administration.

13. A composition for evening dosing comprising a first anti-hypertensive agent, providing on dosing release of said first anti-hypertensive agent followed by the pre-waking release of a second anti-hypertensive agent.

14. The composition of claim 13 wherein said first anti-hypertensive agent is selected from the group consisting of diltiazem and amlodipine.

15. The composition of claim 13 wherein said second anti-hypertensive agent is diltiazem.

16. The composition of claim 15 wherein the weight ratio of the diltiazem is ˜75% to ˜99% of delayed (extended) release (ER) and between ˜1% and ˜25% of immediate release (IR).

17. The composition of claim 13 wherein the pre-waking release said second anti-hypertensive agent occurs about 6-8 h after dosing.

18. The composition of claim 17 wherein said anti-hypertensive agent is selected from the group consisting of an ACE-I and an ARB.

19. The composition of claim 18 wherein said ACE-I is selected from the group consisting of benazepril, lisinopril, enalapril, perindopril erbumine, and ramapril.

20. The composition of claim 18 wherein the ARB is selected from the group consisting of losartan, irbesartan, candesartan, valsartan, eprosartan, telmisartan, and olmesartan.
